(19) United States
(12) Patent Application Publication
(10) Pub. No.: US 2002/0115109 A1
(43) Pub. Date: Aug. 22, 2002

(54) Compositions and methods for diagnosing/treating disease based on beta-catenin/transcription factor interactions

(76) Inventors: Paul Polakis, Mill Valley, CA (US); Bonnee Rubinfeld, Danville, CA (US)

Correspondence Address: ONYX Pharmaceuticals, Inc., 3031 Research Drive, Richmond, CA, 94806, US

(21) Appl. No.: 10/053,422

(22) Filed: Nov. 2, 2001

Related U.S. Application Data
(63) Continuation of application No. 09/035,672, filed on Mar. 5, 1998.
(60) Provisional application No. 60/041,685, filed on Mar. 24, 1997.

(51) Int. Cl. G01N 33/53; C07H 21/04; C12N 9/22

(52) U.S. Cl. 435/7.1; 536/23.2; 435/199

(57) ABSTRACT

Methods and compositions are described that are useful for diagnosing and/or treating disease arising from unwanted cell growth, preferably cancer, involving diagnosing cells for stabilized beta-catenin, or treating cells with compounds that disrupt or alter the formation of a complex consisting of beta-catenin/transcription factor, where the transcription factor is a member of the Lef/Tcf family.

### FIELD OF THE INVENTION

The invention described herein relates generally to the field of human disease, and more specifically to treating and diagnosing disease involving unwanted cell growth based on the identification of compositions of matter that affect beta-catenin interaction with certain transcription factors.

### BACKGROUND OF THE INVENTION

It has been known for some time that a variety of cancers are caused, at least in part, by mutations to certain normal genes, termed “proto-oncogenes.” Proto-oncogenes are involved in regulating normal cell growth in ways that are only now beginning to be appreciated at the molecular level. The mutated proto-oncogenes, or cancer causing genes termed “oncogenes,” disrupt normal cell growth which ultimately causes the death of the organism, if the cancer is not detected and treated in time. During normal or cancer cell growth, proto-oncogenes or oncogenes, are counterbalanced by growth-regulating proteins which regulate or try to regulate the growth of normal or cancer cells, respectively. Such proteins are termed “tumor suppressor proteins,” and include BRCA1, p53, retinoblastoma protein (Rb), adenomatous polyposis coli protein (APC), Wilm's tumor 1 protein (WT1), neurofibromatosis type 1 protein (NF1), and neurofibromatosis type 2 protein (NF2). The interactions of tumor suppressor proteins with other proteins in the cell that regulate their activity is an intense area of biomedical research.

Evidence is accumulating that the protein beta-catenin is associated with certain types of cancers, as well as being an important signaling protein in both Xenopus and Drosophila development (1). Regarding the latter, the proposed pathway, which is initiated by the wnt-1/wingless receptors, involves the post-translational stabilization of b-catenin, leading to its accumulation in the cytoplasm and nucleus. In the nucleus, b-catenin is thought to interact with the LEF/TCF family of transcription factors and thus directly regulate expression of target genes (2). The wnt-1 proto-oncogene also stabilizes b-catenin in mammalian cell culture and promotes tumor formation when expressed in mouse mammary tissue (3).

The potential role of b-catenin signaling in cancer is supported by the observation that the APC tumor suppressor downregulates excess intracellular b-catenin when it is ectopically expressed in colon cancer cells containing defective APC (4). The regulatory mechanism for b-catenin turnover requires the amino-terminal region of the protein. Deletion of this sequence, or mutation of four serine/threonine residues therein, result in the accumulation of b-catenin and thus activate its role in signaling (5, 6, 7). Conceivably, then, mutations that stabilize b-catenin may contribute to loss of cell growth control in tumorigenesis. The identification of these mutations is not presently known, nor is it known how stabilized beta-catenin affects cell growth.

### SUMMARY OF THE INVENTION

A first object of the invention is the description of a family of related isolated nucleic acid sequences that encode stabilized beta-catenin proteins.

A second object of the invention is the description of a substantially pure protein complex consisting of beta-catenin and certain transcription factors, which complex affects cell growth.

A third object of the invention is the description of a substantially pure protein complex consisting of beta-catenin and certain transcription factors, the latter preferably from the Lef/Tcf family of transcription factors.

A fourth object of the invention is the description of a complex consisting of beta-catenin and certain transcription factors, preferably Lef of the family of transcription factors Lef/Tcf which complex affects cell growth.

A fifth object of the invention is the description of methods for identifying compositions of matter that affect the interaction of beta-catenin with certain transcription factors, preferably from the Lef/Tcf family of transcription factors.

A sixth object of the invention is the description of methods of diagnosing or treating disease, preferably those involving unwanted cell growth, including cancer, using compositions of matter that affect the interaction of beta-catenin with certain transcription factors, preferably from the Lef/Tcf family of transcription factors.

These and other objects of the present invention will become apparent to one of ordinary skill in the art upon reading the description of the various aspects of the invention in the following specification. The foregoing and other aspects of the present invention are explained in greater detail in the drawings, detailed description, and examples set forth below.

### BRIEF DESCRIPTION OF THE DRAWINGS

FIG. 1. Analysis of b-catenin and APC in melanoma cell lines. (A) Protein-equivalent amounts of total cell lysate from the indicated cell lines were subjected to SDS-polyacrylamide gel electrophoresis (PAGE) and immunoblotting (13). The blot was cut horizontally and developed with anti-APC2 (top) or anti-b-catenin (bottom). The b-catenin blot was developed with ¹²⁵I-protein A and the counts per minute (CPM) for each b-catenin band is indicated below each lane. (B) APC was immunoprecipitated from protein-equivalent amounts of the cell lysates and the precipitates analyzed for APC and b-catenin by SDS-PAGE and immunoblotting (13). Values at left indicate positions and molecular masses in kilodaltons of protein standards. NHEM indicates a normal neonatal human melanocyte. All other cell lines were derived from human melanomas (16). (C) Size exclusion chromatography was performed on approximately 800 mg total protein from each lysate and fractions were analyzed for b-catenin by SDS-PAGE and immunoblotting. Total lysate (L) and column fraction (FRX.) numbers are shown at top, and arrows indicate the elution positions of protein standards. Longer exposures are presented for cell lines with lower levels of total b-catenin.

FIG. 2. Downregulation of b-catenin by ectopic expression of WT APC. The 928 mel and 888 mel cells were transiently transfected with a plasmid encoding human WT APC and 48 hours later, cells were fixed and costained with anti-APC (left) and anti-b-catenin (right) (18).

FIG. 3. Pulse-chase analysis of b-catenin. (A) Melanoma cells were pulse-labeled with ³⁵S-methionine, chased with cold methionine for the indicated times, and then lysed (20). Beta-catenin was immunoprecipitated and analyzed by SDS-PAGE and fluorography. The cell lines are indicated to the left of each panel at the position of the b-catenin band. DN indicates the position of the amino-terminal truncated form of b-catenin in the 1088 mel cells. (B) ATT20 cell lines stably expressing either wildtype b-catenin (wt) or the ser37ala mutant (S37A) were subjected to pulse-chase analysis (20). (C) SW480 cells were transiently cotransfected with plasmids encoding a carboxy-terminal (APC3) or central (APC25) fragment of APC and either the WT or ser37ala mutant of b-catenin (20). APC25 downregulates b-catenin but APC3 does not (4).

FIG. 4. Coimmunoprecipitation of LEF1 with b-catenin. Beta-catenin was immunoprecipitated from ˜600 mg total protein from the indicated cell lysates and the precipitates analyzed for b-catenin and LEF1 by SDS-PAGE and immunoblotting (13).

### DETAILED DESCRIPTION OF THE INVENTION

All publications and patent applications mentioned in this specification are herein incorporated by reference to the same extent as if each individual publication or patent application was specifically and individually indicated to be incorporated by reference.

Definitions

At the outset it is worth noting that unless defined otherwise, all technical and scientific terms used herein have the same meaning as commonly understood by one of ordinary skill in the art to which this invention belongs. Generally, the nomenclature used herein and the laboratory procedures described below are those well known and commonly employed in the art. Standard techniques are used for recombinant nucleic acid methods, polynucleotide synthesis, and microbial culture and transformation (e.g., electroporation, lipofection). Generally enzymatic reactions and purification steps are performed according to the manufacturer's specifications. The techniques and procedures are generally performed according to conventional methods in the art and various general references (see generally, Sambrook et al., Molecular Cloning: A Laboratory Manual, 2nd. edition (1989) Cold Spring Harbor Laboratory Press, Cold Spring Harbor, N.Y., which is incorporated herein by reference) which are provided throughout this document. The nomenclature used herein and the laboratory procedures in analytical chemistry, organic synthetic chemistry, and pharmaceutical formulation described below are those well known and commonly employed in the art. Standard techniques are used for chemical syntheses, chemical analyses, pharmaceutical formulation and delivery, and treatment of patients. In the formulas representing selected specific embodiments of beta-catenin or transcription factors of the present invention, the amino-and carboxy-terminal groups, although often not specifically shown, will be understood to be in the form they would assume at physiological pH values, unless otherwise specified. Thus, the N-terminal H₂⁺ and C-terminal-O⁻ at physiological pH are understood to be present though not necessarily specified and shown, either in specific examples or in generic formulas. In the polypeptide notation used herein, the left-hand end of the molecule is the amino terminal end and the right-hand end is the carboxy-terminal end, in accordance with standard usage and convention. Of course, the basic and acid addition salts including those which are formed at nonphysiological ph values are also included in the compounds of the invention. The amino acid residues described herein are preferably in the “L” isomeric form. Stereoisomers (e.g., D-amino acids) of the twenty conventional amino acids, unnatural amino acids such as a,a-distributed amino acids, N-alkyl amino acids, lactic acid, and other unconventional amino acids may also be suitable components for polypeptides of the present invention, as long as the desired functional property is retained by the polypeptide. For the peptides shown, each encoded residue where appropriate is represented by a three letter designation, corresponding to the trivial name of the conventional amino acid, in keeping with standard polypeptide nomenclature (described in J. Biol. Chem., 243:3552-59 (1969) and adopted at 37 CFR §1.822(b)(2)).

Free functional groups, including those at the carboxy- or amino-terminus, referred to as noninterfering substituents, can also be modified by amidation, acylation or other substitution, which can, for example, change the solubility of the compounds without affecting their activity.

As employed throughout the disclosure, the following terms, unless otherwise indicated, shall be understood to have the following meanings:

The term “isolated protein” referred to herein means a protein of cDNA, recombinant RNA, or synthetic origin or some combination thereof, which by virtue of its origin the “isolated protein” (1) is not substantially associated with proteins found in nature, (2) is substantially free of other proteins from the same source, e.g. free of human proteins, (3) may be expressed by a cell from a different species, or (4) does not occur in nature.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset with 200 bases or fewer in length. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides. The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. An oligonucleotide can include a label for detection, if desired.

The term “sequence homology” referred to herein describes the proportion of base matches between two nucleic acid sequences or the proportion amino acid matches between two amino acid sequences. When sequence homology is expressed as a percentage, e.g., 50%, the percentage denotes the proportion of matches over the length of sequence from beta-catenin or Lef that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and most preferably not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof are more preferably homologous if their amino acids are greater than or equal to 50% identical when optimally aligned using the ALIGN program.

As used herein, “substantially pure” means an object species is the predominant species present (i.e., on a molar basis it is more abundant than any other macromolecular individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The phrase “stabilized beta-catenin” is meant to include those compositions of matter as set forth and discussed below. It will be appreciated, however, by the skilled practitioner of this art that in many instances where reference to “stabilized beta-catenin” is made, particularly in an assay format context, that wild type beta-catenin can be substituted. Indeed, in most of the b-catenin/Lef assays aimed at identifying compositions of matter that affect this complex or its formation, wild type beta-catenin will substitute for “stabilized beta-catenin.”

Chemistry terms herein are used according to conventional usage in the art, as exemplified by The McGraw-Hill Dictionary of Chemical Terms (ed. Parker, S., 1985), McGraw-Hill, San Francisco, incorporated herein by reference.

The production of proteins from cloned genes by genetic engineering is well known. See, e.g. U.S. Pat. No. 4,761,371 to Bell et al. at column 6, line 3 to column 9, line 65. (The disclosure of all patent references cited herein is to be incorporated herein by reference.) The discussion which follows is accordingly intended as an overview of this field, and is not intended to reflect the full state of the art.

DNA regions are operably linked when they are functionally related to each other. For example: a promoter is operably linked to a coding sequence if it controls the transcription of the sequence; a ribosome binding site is operably linked to a coding sequence if it is positioned so as to permit translation. Generally, operably linked means contiguous and, in the case of leader sequences, contiguous and in reading frame.

Suitable host cells include prokaryotes, yeast cells, or higher eukaryotic cells. Prokaryotes include gram negative or gram positive organisms, for example Escherichia coli (E. coli) or Bacilli. Higher eukaryotic cells include established cell lines of mammalian origin as described below. Exemplary host cells are DH5a, E. coli W3110 (ATCC 27,325), E coli B, E. coli X1776 (ATCC 31,537) and E. coli 294 (ATCC 31,446). Pseudomonas species, Bacillus species, and Serratia marcesans are also suitable.

In an insect system, Autographa californica nuclear polyhidrosis virus (AcNPV) may be used as a vector to express foreign genes. (E.g., see Smith et al., 1983, J. Virol. 46: 584; Smith, U.S. Pat. No. 4,215,051). Sf9 insect cells can be infected with a baculovirus vector expressing a glu-glu epitope tagged beta-catenin construct. See, Rubinfeld, et al., J. Biol. Chem. vol. 270, no. 10, pp 5549-5555 (1995). Other epitope tags may be employed that are known in the art including a 6×histidine tag, myc, or an EE-tag (i.e. Glu—Glu-tag). “E” refers to the amino acid glutamine.

A broad variety of suitable microbial vectors are available. Generally, a microbial vector will contain an origin of replication recognized by the intended host, a promoter which will function in the host and a phenotypic selection gene such as a gene encoding proteins conferring antibiotic resistance or supplying an autotrophic requirement. Similar constructs will be manufactured for other hosts. E. coli is typically transformed using pBR322. See Bolivar et al., Gene 2, 95 (1977). pBR322 contains genes for ampicillin and tetracycline resistance and thus provides easy means for identifying transformed cells. Expression vectors should contain a promoter which is recognized by the host organism. This generally means a promoter obtained from the intended host. Promoters most commonly used in recombinant microbial expression vectors include the beta-lactamase (penicillinase) and lactose promoter systems (Chang et al., Nature 275, 615 (1978); and Goeddel et al., Nucleic Acids Res. 8, 4057 (1980) and EPO Application Publication Number 36,776) and the tac promoter (H. De Boer et al., Proc. Natl. Acad. Sci. USA 80, 21 (1983)). While these are commonly used, other microbial promoters are suitable. Details concerning nucleotide sequences of many promoters have been published, enabling a skilled worker to operably ligate them to DNA encoding beta-catenin in plasmid or viral vectors (Siebenlist et al., Cell 20, 269, 1980)). The promoter and Shine-Dalgarno (SD) sequence (for prokaryotic host expression) are operably linked to the DNA encoding beta-catenin, i.e. they are positioned so as to promote transcription of the beta-catenin messenger RNA from the DNA. The SD sequence is thought to promote binding of mRNA to the ribosome by the pairing of bases between the SD sequence and the 3′ end of E. coli 16S rRNA (Steitz et al. (1979). In Biological Regulation and Development: Gene Expression (ed. R. F. Goldberger)). To express eukaryotic genes and prokaryotic genes with a weak ribosome-binding site see Sambrook et al. (1989) “Expression of cloned genes in Escherichia coli.” In Molecular Cloning: A Laboratory Manual. Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system (Studier et al. (1986) J. Mol. Biol. 189:113; Tabor et al. (1985) Proc. Natl. Acad. Sci. 82:1074). In addition, a hybrid promoter can also be composed of a bacteriophage promoter and an E. coli operator region (EPO Pub. No. 267,851).

Stabilized beta-catenin, or wild type beta-catenin can be expressed intracellularly. A promoter sequence can be directly linked with a beta-catenin gene or a fragment thereof, in which case the first amino acid at the N-terminus will always be a methionine, which is encoded by the ATG start codon. If desired, methionine at the N-terminus can be cleaved from the protein by in vitro incubation with cyanogen bromide or by either in vivo on in vitro incubation with a bacterial methionine N-terminal peptidase (EPO Pub. No. 219,237).

Eukaryotic microbes such as yeast cultures may be transformed with suitable beta-catenin vectors. See, e.g. U.S. Pat. No. 4,745,057. Saccharomyces cerevisiae is the most commonly used among lower eukaryotic host microorganisms, although a number of other strains are commonly available. Yeast vectors may contain an origin of replication from the 2 micron yeast plasmid or an autonomously replicating sequence (ARS), a promoter, DNA encoding beta-catenin, sequences for polyadenylation and transcription termination, and a selection gene.

Suitable promoting sequences in yeast vectors include the promoters for metallothionein, 3-phosphoglycerate kinase (Hitzeman et al., J. Biol. Chem. 255, 2073 (1980) or other glycolytic enzymes (Hess et al., J. Adv. Enzyme Reg. 7, 149 (1968); and Holland et al., Biochemistry 17, 4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promotes for use in yeast expression are further described in R. Hitzman et al., EPO Publication Number 73,657.

Cultures of cells derived from multicellular organisms are a desirable host for recombinant beta-catenin synthesis. In principal, any higher eukaryotic cell culture is workable, whether from vertebrate or invertebrate culture. However, mammalian cells are preferred, as illustrated in the Examples. Propagation of such cells in cell culture has become a routine procedure. See Tissue Culture, Academic Press, Kruse and Paterson, editors (1973).

The transcriptional and translational control sequences in expression vectors to be used in transforming vertebrate cells are often provided by viral sources. For example, commonly used promoters are derived from CMV, polyoma, Adenovirus 2, and Simian Virus 40 (SV40). See, e.g., U.S. Pat. No. 4,599,308.

An origin of replication may be provided either by construction of the vector to include an exogenous origin, such as may be derived from SV40 or other viral source (e.g. Polyoma, Adenovirus, VSV, or BPV), or may be provided by the host cell chromosomal replication mechanism. If the vector is integrated into the host cell chromosome, the latter may be sufficient.

Identification of Stabilized Beta-Catenin

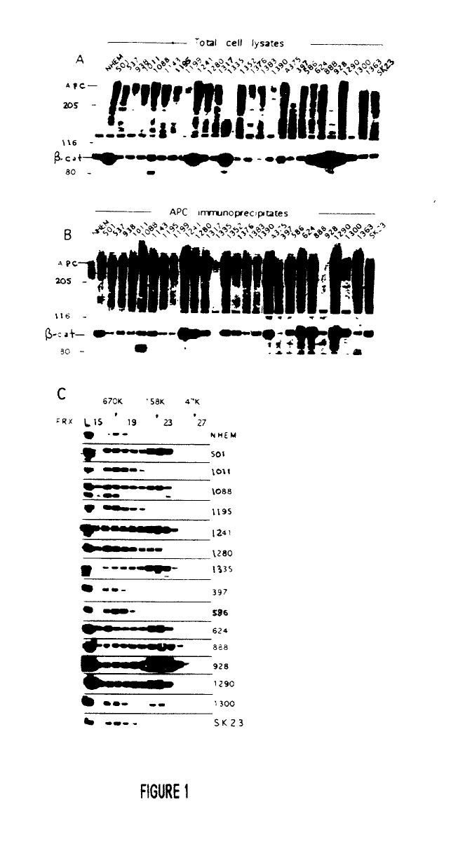

Previously, a mutant form of b-catenin, containing a ser³⁷ - phe³⁷ substitution, was identified in the 888 mel cell line as a melanoma-specific antigen recognized by tumor infiltrating lymphocytes (8). As it was possible that this mutation increased the stability of b-catenin, we determined b-catenin levels in these cells and in 25 other melanoma cell lines. Seven of the lines including the 888 mel cell, contained elevated amounts of b-catenin relative to normal human neonatal melanocytes (NHEM) (FIG. 1A). Two of the seven appeared to have APC alterations as well: the 1335 mel cells contained a truncated APC and the 928 mel cells had no detectable APC. The truncated APC was not immunoprecipitated by antibody specific to carboxy-terminal sequence of APC, suggesting it was a carboxy-terinal truncation similar to that observed in colon cancers (FIG. 1B).

Substantial amounts of b-catenin was coimmunoprecipitated with wild-type (WT) APC from five other lines with high levels of b-catenin. The accumulation of b-catenin on WT APC is characteristic of b-catenin stabilization, as has been observed in particular with amino-terminal deletion mutants of b-catenin (5). The 1088 mel cell appeared to contain a truncated b-catenin that accumulated on the APC protein. Another characteristic of stabilized b-catenin is its migration in a monomeric pool upon size fractionation chromatography (5, 9, 10). All of the melanoma cells with elevated levels of b-catenin exhibited a substantial pool of monomeric b-catenin (FIG. 1C). In addition, two of the cell lines with normal levels of b-catenin, the 1280 and 1300 mel, also contained some monomeric b-catenin.

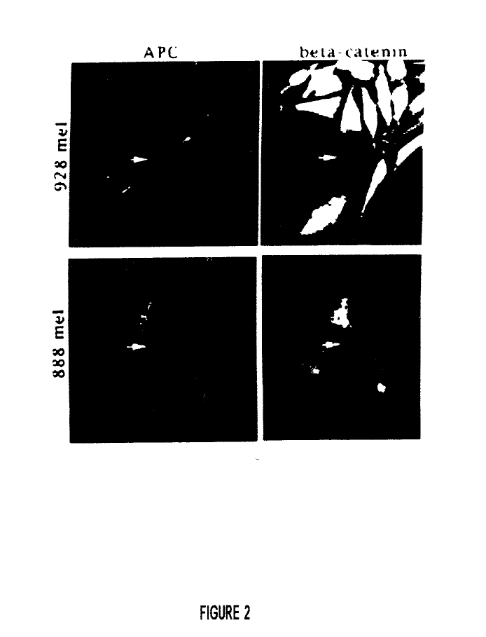

Upregulation of b-catenin in the 928 and 1335 mel cell lines may have resulted from loss of WT APC, as has been proposed for colon cancer cells (4). To test this hypothesis, we transiently expressed WT APC in the 928 mel cells and costained them with antibodies specific to APC and b-catenin. The 928 mel cells that were positive for ectopically expressed APC contained low levels of b-catenin relative to nontransfected cells, which exhibited excessive nuclear and cytoplasmic staining (FIG. 2). The ability of APC to downregulate b-catenin in the 928 mel cells suggested they contained WT b-catenin. By contrast, ectopic expression of WT APC in the 888 mel cells did not downregulate the endogenous mutant b-catenin, but instead resulted in its accumulation on the WT APC.

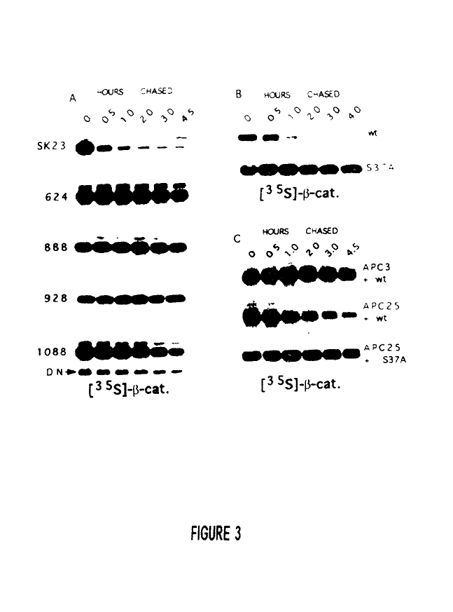

The wnt-1 proto-oncogene activates b-catenin signaling by reducing the rate of b-catenin degradation (3), whereas the APC tumor suppressor enhances this rate (4). To examine whether the high steady state level of b-catenin in the melanoma cells was due to a reduced rate of turnover, we performed pulse-chase analysis of b-catenin on representative cell lines. The b-catenin in the SK23 mel cell line, which contains WT APC and normal levels of b-catenin, had a half-life (t^{½}) of less than 30 min (FIG. 3A). By contrast, the b-catenin in the 888 mel cells, which contained the ser³⁷phe mutation, had a t^{½} of over 4.5 hours. The b-catenin in the 928 mel cells, which lack WT APC, and in the 624 mel cells, which contain a mutant b-catenin (Table 1), also had an extended t^{½}. The 888 mel cells contain mRNAs for both wildtype and mutant b-catenins (8), but the relative contribution of their products to the half-life analysis is unknown. The results suggest that the WT b-catenin is a minor fraction of the total or that the mutant form dominantly interferes with the turnover of the WT protein. The 1088 mel cells contain both a full-length b-catenin with an intermediate t^{½} of ˜2 hours, and a truncated b-catenin with an extended half-life of greater than 4.5 hours.

To ensure that substitution of ser³⁷ was responsible for the reduced rate of protein turnover, we transfected murine pituitary ATT20 cells, which exhibit rapid turnover of endogenous b-catenin (5), with plasmids encoding epitope-tagged ser^{37 - - -} ala³⁷ or WT b-catenin. The exogenous WT b-catenin had a t^{½} of ˜40 minutes, whereas the ser³⁷ - - - ala³⁷ b-catenin had a t^{½} of greater than 4 hours (FIG. 3B). To determine if the ser³⁷ - - - ala³⁷ b-catenin was responsive to APC-dependent turnover, we coexpressed it with an APC25 CDNA in SW480 human colon cancer cells which contain only truncated APC. The APC25 fragment downregulates b-catenin, whereas the control APC3 fragment does not (4). Recovery of the epitope tagged b-catenins revealed that WT, but not the ser³⁷ - - - ala³⁷ b-catenin, was degraded in response to the coexpressed APC25 fragment (FIG. 3C). These results demonstrate that a single point mutation has a dramatic effect on the t^{½} of b-catenin.

Sequencing of b-catenin cDNAs from the other melanoma lines with b-catenin accumulation revealed three additional point mutations affecting serine residues (Table 1).

TABLE 1
Beta-catenin mutations in melanoma cell lines.

| Cell line | Nucleotide | Protein |
|---|---|---|
| 501 mel | TCT to TTT | ser37phe |
| 1088 mel | mRNA del.exons 2 and 3 | del. a.a.1-87¹ |
| | mRNA del.exons 2, 3 and 4 | del. a.a.1-173¹ |
| 1241 mel | TCT to TTT | ser37phe_ |
| 1335 mel² | wild type | wild type |
| 624 mel | TCT to TAT | ser45tyr |
| 888 mel | TCT to TTT | ser37phe |
| 928 mel² | N.D.³ | |
| 1290 mel | TCT to TTT | ser37phe |

¹Minimum deletion of amino acid sequence based on reinitiation at next nearest methionine codon.
²These cells lack wildtype APC protein.
³Not determined.

As with the 888 mel cells, the mutations identified in the 501 and 1241 mel cells were C to T transitions that produced a S37F substitution. Interestingly, C to T transitions are also common in the p53 gene in melanomas, and may be an effect of ultraviolet radiation (11). The mutation in 624 mel predicts ser⁴⁵ - - - tyr⁴⁵ substitution and pulse-chase analysis of this cell suggests that it may prolong the t^{½} of b-catenin (FIG. 3). Moreover, coexpression of a S45Y b-catenin with APC25 indicated it was refractory to APC-dependent turnover in SW480 cells (12). The serines 37 and 45 are likely important phosphorylation sites, as the quadruple substitution of ser33, ser37, thr4 and ser45 markedly reduced the phosphorylation of b-catenin in Xenopus (7). Two novel b-catenin mRNAs, one lacking exons 2 and 3, and the other lacking exons 2, 3 and 4, were identified in the 1088 mel cells. Initiation normally occurs at codon 1 in exon 2, however, initiation at codon 88, the first ATG in exon 4, would account for a truncated b-catenin approximately the size of that detected in the 1088 mel cells (FIG. 1A). A more severely truncated b-catenin, predicted from initiation at codon 174 in exon 5 of the other alternative mRNA, has not been detected. Whether the b-catenin mRNA isoforms in this cell are due to a mutation or to unusual mRNA processing is unclear. None of the other melanoma cells contained these mRNAs. Sequencing of b-catenin cDNAs from the APC-deficient 1335 and 928 mel cells identified only wild-type sequence, as did sequencing of the 1280 mel, 1300 mel, SK23 mel, and NHEM lines.

Interaction of Stabilized Beta-Catenin with Transcription Factors

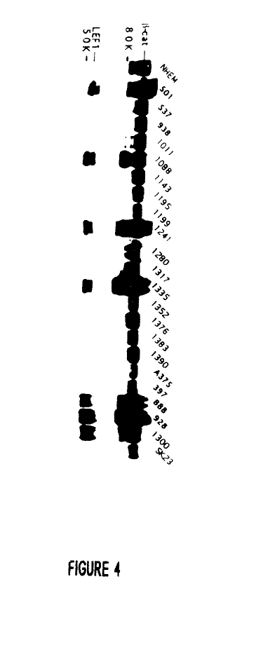

Recently, b-catenin has been show to functionally interact with LEF/TCF transcription factors when overexpressed in Xenopus oocytes (2). To determine if this interaction occurs in the melanoma cell lines, we immunoprecipitated b-catenin from some of the lines and examined the precipitates for LEF1. LEF1 was preferentially coimmunoprecipitated by anti-b-catenin from the cells containing stabilized b-catenin (FIG. 4). This indicates that in these cells a constitutive b-catenin-LEF/TCF complex results in persistent transactivation of as yet unidentified target genes, causing unwanted cell growth, or cancer.

Of the 26 melanoma cell lines examined here, 8 are defective in b-catenin regulation, because of b-catenin mutations, unusual b-catenin mRNA splicing, or inactivation of APC. We hypothesize that these mutations are selected in tumor progression. The mutation in the 888 mel line was unlikely to be generated by in vitro culture, as it was also present in the 1290 mel line, which was derived from a new tumor from the same patient after a three-year remission (8). Moreover, the mutation was also identified in the uncultured tumor material from which the 1290 mel was derived. The stabilizing mutations in b-catenin are also consistent with a proposed function for APC in colon cancer. The ability of WT, but not mutant APC to downregulate b-catenin in colon cancer cells, supports the work described herein that upregulation of b-catenin contributes to cancer progression (4). In the melanoma cells, b-catenin mutations were identified in cells that appeared to express WT APC, whereas high levels of WT b-catenin was found in cells expressing mutant APC. Thus, upregulation of b-catenin is be a common feature of tumorigenesis that is effected through mutations in the APC or b-catenin genes or other genes that function in this pathway.

Screening Assays for Compounds that Modulate Stabilized Beta-Catenin Expression or Activity

The following assays are designed to identify compounds that interact with (eg., bind to) stabilized beta-catenin or Lef, to affect the binding of stabilized beta-catenin to Lef, compounds that interact with (e.g., bind to) intracellular proteins that interact with stabilized beta-catenin and/or Lef, compounds that interfere with the interaction of stabilized beta-catenin with Lef or with other transcription factors that mediate beta-catenin activity, and to compounds which modulate the activity of the stabilized beta-catenin gene (i.e., modulate the level of stabilized beta-catenin gene expression) or modulate the level of stabilized beta-catenin. Assays may additionally be utilized which identify compounds which bind to stabilized beta-catenin gene regulatory sequences (eg., promoter sequences) and which may modulate stabilized beta-catenin gene expression. See e.g., Platt, K. A., 1994, J. Biol. Chem. 269:28558-28562, which is incorporated herein by reference in its entirety.

The compounds which may be screened in accordance with the invention include but are not limited to peptides, antibodies and fragments thereof, prostaglandins, lipids and other organic compounds (i.e., terpines, peptidoniimetics) that bind to stabilized beta-catenin or Lef and either mimic the activity triggered by the natural ligand (i.e., agonists) or inhibit the activity triggered by the natural ligand (i.e., antagonists); as well as peptides, antibodies or fragments thereof, and other organic compounds that mimic stabilized beta-catenin or Lef (or a portion thereof).

Such compounds may include, but are not limited to, peptides such as, for example, soluble peptides, including but not limited to members of random peptide libraries (see, e.g., Lam, K. S. et al., 1991, Nature 354:82-84; Houghten, R. et al., 1991, Nature 354:84-86), and combinatorial chemistry-derived molecular library peptides made of D- and/or L-configuration amino acids, phosphopeptides (including, but not limited to members of random or partially degenerate, directed phosphopeptide libraries; see, e.g., Songyang, Z. et al., 1993, Cell 72:767-778); antibodies (including, but not limited to, polyclonal, monoclonal, humanized, anti-idiotypic, chimeric or single chain antibodies, and FAb, F(ab.)₂ and FAb expression library fragments, and epitope-binding fragments thereof); and small organic or inorganic molecules.

Other compounds which can be screened in accordance with the invention include but are not limited to small organic molecules that are able to gain entry into an appropriate cell and affect the expression of the stabilized beta-catenin gene or some other gene involved in the stabilized beta-catenin signal transduction pathway (e.g., by interacting with the regulatory region or transcription factors involved in gene expression); or such compounds that affect the activity of the stabilized beta-catenin, e.g., by inhibiting or enhancing the binding of stabilized beta-catenin to Lef or the binding of stabilized beta-catenin to some other transcription factor involved in the stabilized beta-catenin signal transduction pathway.

Computer modeling and searching technologies permit identification of compounds, or the improvement of already identified compounds, that can modulate stabilized beta-catenin or Lef expression or activity. Having identified such a compound or composition, the binding sites or regions are identified. Such binding sites might typically be the binding partner sites, such as, for example, the interaction domains of the Lef with stabilized beta-catenin itself. The binding site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the binding site by finding where on the factor the complexed ligand is found.

Next, the three dimensional geometric structure of the binding site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the binding site structure determined.

If an incomplete or insufficiently accurate structure is determined, the methods of computer based numerical modelling can be used to complete the structure or improve its accuracy. Any recognized modelling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the binding region(s) of beta-catenin or Lef, either experimentally, by modeling, or by a combination, candidate modulating compounds can be identified by searching databases containing compounds along with information on their molecular structure. Such a search seeks compounds having structures that match the determined binding site structure and that interact with the groups defining the site(s) Such a search can be manual, but is preferably computer assisted. These compounds found from this search are potential stabilized beta-catenin modulating compounds.

Alternatively, these methods can be used to identify improved modulating compounds from an already known modulating compound or ligand. The composition of the known compound can be modified and the structural effects of modification can be determined using the experimental and computer modelling methods described above applied to the new composition. The altered structure is then compared to the binding site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the binding sites of stabilized beta-catenin that interact with Lef, and related transduction and transcription factors will be apparent to those of skill in the art.

Examples of molecular modeling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modelling of drugs interactive with specific proteins, such as Rotivinen et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure—Activity Relationships in Drug Design pp.189-193 (Alan R. Liss, Inc. 1989); Lewis and Dean, 1989, Proc. R. Soc. Lond. 236:125-140 and 141-162; and, with respect to a model receptor for nucleic acid components, Askew et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elaborating the biological function of the stabilized beta-catenin gene product, and for ameliorating hematopoietic lineage cell activation disorders. Assays for testing the effectiveness of compounds, identified by techniques described herein are discussed below.

In vitro Screening Assays for Compounds that Bind to Beta-Catenin

In vitro systems may be designed to identify compounds capable of interacting with (e.g., binding to) stabilized beta-catenin and/or transcription factors that bind beta-catenin, preferably Lef. Compounds identified may be useful, for example, in modulating the activity of wild type and/or mutant stabilized beta-catenin gene products; may be utilized in screens for identifying compounds that disrupt normal stabilized beta-catenin/Lef interactions; or may in themselves disrupt such interactions.

The principle of the assays used to identify compounds that bind to the stabilized beta-catenin involves preparing a reaction mixture of the stabilized beta-catenin and the test compound under conditions and for a time sufficient to allow the two components to interact and bind, thus forming a complex which can be removed and/or detected in the reaction mixture. The stabilized beta-catenin species used can vary depending upon the goal of the screening assay. For example, the full length stabilized beta-catenin, or a fusion protein containing the stabilized beta-catenin fused to a protein or polypeptide that affords advantages in the assay system (e.g., labeling, isolation of the resulting complex, etc.) can be utilized.

The screening assays can be conducted in a variety of ways. For example, one method to conduct such an assay would involve anchoring the stabilized beta-catenin protein, polypeptide, peptide or fusion protein or the test substance onto a solid phase and detecting stabilized beta-catenin/test compound complexes anchored on the solid phase at the end of the reaction. In one embodiment of such a method, the stabilized beta-catenin reactant may be anchored onto a solid surface, and the test compound, which is not anchored, may be labeled, either directly or indirectly. In another embodiment of the method, a stabilized beta-catenin protein anchored on the solid phase is complexed with labeled Lef. Then, a test compound could be assayed for its ability to disrupt the association of the stabilized beta-catenin/Lef complex.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for stabilized beta-catenin protein, polypeptide, peptide or fusion protein, or the Lef protein or fusion protein, or the test compound to anchor any complexes formed in solution, and a labeled antibody specific for the other component of the possible complex to detect anchored complexes.

Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Diagnostic Applications

It will be apparent to the skilled practitioner of this art that any method that allows for the detection of stabilized beta-catenin, either beta-catenin protein or nucleic acid, can be used as a diagnostic method for unwanted cell growth, including cancer. Such methods would include antibody or nucleic acid based assays.

References

THE FOLLOWING REFERENCES AND NOTES ARE REFERRED TO THROUGHOUT THE SPECIFICATION ABOVE.

1. B. M. Gumbiner, Curr. Opin. Cell Biol. 7, 634 (1995); M. Peifer, Trends Cell Biol. 5, 224 (1995); J. Klingensmith and R. Nusse, Dev. Biol. 166, 396 (1994).

2. J. Behrens et al., Nature 382, 638 (1996); M. Molenaar et al., Cell 86, 391 (1996); Huber et al., Mech. Dev. 59, 3 (1996)

3. R. S. Bradley, P. Cowin, A. M. C. Brown, J. Cell. Biol. 123, 1857 (1993); L. Hinck, W. J. Nelson, J. Papkoff, J. Cell. Biol. 124,729 (1994); A. S. Tsukamoto, R. Grosschedl, R. C. Guzman, T. Parslow, H. E. Varmus, Cell 55, 619 (1988).

4. S. Munemitsu, I. Albert, B. Souza, B. Rubinfeld, P. Polakis, Proc. Natl. Acad. Sci. U.S.A. 92, 3046 (1995).

5. S. Munemitsu, I. Albert, B. Rubinfeld, P. Polakis, Mol. Cell. Biol. 16, 4088 (1996).

6. N. Funayama, F. Fagotto, P. McCrea, B. M. Gumbiner, J. Cell. Biol. 128, 959 (1995); S. Orsulic, M. Peifer, J. Cell. Biol. 134, 1283 (1996).

7. C. Yost et al., Genes Dev. 10, 1443 (1996).

8. P. F. Robbins et al., J. Exp. Med. 183, 1185 (1996).

9. J. Papkoff, B. Rubinfeld, B. Schryver, P. Polakis, Mol. Cell. Biol. 16, 2128 (1996).

10. This monomeric pool represents unbound b-catenin, but does not reflect a lack of association of b-catenin with its binding proteins. For example, cells with this pool of excess b-catenin generally have much higher amounts of b-catenin associated with APC than those without. There is 100-1000-fold molar excess of b-catenin over APC in most cells and, therefore, saturation of APC with b-catenin would not significantly deplete the monomeric b-catenin pool.

11. D. E. Brash et al., Proc. Natl. Acad. Sci. U.S.A. 88, 10124 (1991).

12. B. Rubinfeld, P. Polakis, unpublished results.

13. Cell pellets were lysed in Triton X-100 lysis buffer [20 mM tris-HCl (pH 8.0), 1.0% Triton X-100, 140 mM NaCl, 10% glycerol, 1 mM EGTA, 1.5 mM MgCl₂, 1 mM dithiothreitol (DTT), 1 mM sodium vanadate, 50 mM NaF, 1 mM Pefabloc, 10 mg/ml each of Aprotinin, pepstatin and leupeptin] and after centrifugation the supernatants were adjusted to 2 mg/ml total protein. Twenty five ml of each sample was applied to 6% SDS-polyacrylamide gel for analysis of total b-catenin and APC by immunoblotting. For immunoprecipitations, 400 ml of each lysate was incubated with 2 mg of affinity-purified polyclonal b-catenin antibody or 2 mg affinity purified polyclonal APC3 antibody(14). Antibodies were recovered using Protein A Sepharose and the beads were washed three times with 1 ml each of buffer B [20 mM tris-HCl (pH8.0), 150 mM NaCl, 0.5% NP40] and finally eluted with SDS-PAGE sample buffer. For immunoblotting, affinity-purified rabbit polyclonal antibody raised against the central region of APC (APC2), full length b-catenin or full-length LEF1 (15) were incubated with the blots at 0.2 mg/ml. Blots were developed using either the ECL system (Amersham) or, for the b-catenin blot in FIG. 1A, ¹²⁵I-protein A at 0.5 mCi/ml (Amersham).

14. B. Rubinfeld et al., Science 262, 1731 (1993).

15. M. G. Prieve, K. L. Guttridge, J. E. Munguia, M. L. Waterman, J. Biol. Chem. 271, 7654 (1996).

16. The melanoma cell lines were generated from metastatic lesions (17) with the exception of the SK23 mel (21). The 888 and 1290 mel lines were derived from two independent metastases from the same patient, all others originated from separate patients. The SW480 cell line was obtained from the American Type Culture Collection (ATCC reference CCL228) and is a human colon cancer cell line. ATT20 (ATCC reference CCL89) is a murine pituitary tumor cell. Stable ATT20 clones expressing b-catenins were generated as previously described in (5).

17. S. Topalian, D. Solomon, S. Rosenberg, J. Immunol. 142, 3714 (1989).

18. Transient transfection of 928 mel cells with plasmid encoding human WT APC was performed using lipofectamine (BRL) (4). Cells were fixed 48 hours later and stained for imnnunofluorescence microscopy (19). For detection of APC, cells were first incubated with carboxy-terminal specific APC3 antibody (14), and then with FITC-conjugated goat antibody to rabbit immunoglobin G (IgG) (Sigma). Beta-catenin was detected with mouse anti-b-catenin Mab (Transduction Laboratories, Lexington, Ky.) and Texas red-conjugated donkey antibody to mouse IgG (Cappel, Durham, N.C.).

19. S. Munemitsu et al., Cancer Res. 54, 3676 (1994).

20. Cells were pulse-labeled (4) for 30 min and then incubated with media containing unlabeled methionine for the indicated times prior to lysis on the culture dish. After centrifugation of the lysates, b-catenin was immunoprecipitated from the melanoma cell supernatants with anti-b-catenin, and from the ATT20 or SW480 supernatants by antibody to myc that had been covalently coupled to protein G Sepharose. Immunoprecipitates were subjected to electrophoresis and fluorography on 8% SDS-polyacrylamide gels. In the transfection experiments (4), greater than 50% of the SW480 cells expressed the ectopic cDNA. The APC25 construct encodes APC amino acids 1034 to 2130 and APC3 encodes amino acids 2130 to 2843. For isolation of b-catenin cDNAs, a cDNA pool was first obtained by reverse transcription of total mRNA (RNeasy kit, Qiagen) using a mixture of oligo-dT and random primers. PCR was then performed on the cDNA pool using six distinct primer sets specific for b-catenin cDNA, and the PCR products were cloned into pCR2.1 (Invitrogen) and propagated in E. Coli. Beta-catenin mutations were confirmed by sequencing analysis of PCR products obtained with the multiple primer sets.

21. M. Waterman provided antibody to LEF1, and T. Boon the SK23 mel cells.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

We claim:

1. Isolated substantially purified stabilized beta-catenin protein, or fragments thereof.

2. Isolated DNA that encodes substantially purified stabilized beta-catenin protein, or fragments thereof.

3. Isolated DNA that encodes substantially purified stabilized beta-catenin protein, or fragments thereof, wherein said isolated DNA is cDNA.

4. An isolated substantially purified protein complex comprising purified stabilized beta-catenin protein, or fragments thereof, and a transcription factor.

5. An isolated substantially purified protein complex as described in claim 4, wherein said transcription factor is a member of the Lef/Tcf family.

6. A method of diagnosing for disease based on unwanted cell growth comprising determining the presence of stabilized beta-catenin in said cells.

7. A method as described in claim 6 wherein said disease is cancer.

8. A method as described in claim 6 wherein said cancer is melanoma.

9. A method of identifying compounds that inhibit unwanted cell growth comprising assaying for compounds that prevent the formation of a complex comprising beta-catenin and a transcription factor.

10. A method of identifying compounds that inhibit unwanted cell growth as described in claim 9 wherein said transcription factor is a member of the Lef/Tcf family.

11. A method of identifying compounds that inhibit unwanted cell growth as described in claim 10 wherein said transcription factor is Lef.
